(19) United States
(12) Patent Application Publication
(10) Pub. No.: US 2010/0129826 A1
(43) Pub. Date: May 27, 2010

(54) Double stranded RNA receptor (dsRNA-R) and methods relating thereto

(76) Inventor: Mary Lee Mackichan, San Francisco, CA (US)

Correspondence Address: NOVARTIS VACCINES AND DIAGNOSTICS INC., INTELLECTUAL PROPERTY- X100B, P.O. BOX 8097, Emeryville, CA, 94662-8097, US

(21) Appl. No.: 12/657,157

(22) Filed: Jan. 14, 2010

Related U.S. Application Data
(63) Continuation of application No. 11/350,151, filed on Feb. 8, 2006, now abandoned.
Application No. 11/350,151 is a continuation of application No. 10/129,158, filed on Jan. 29, 2003, now abandoned.
(60) Provisional application No. 60/163,157, filed on Nov. 2, 1999.
(60) Provisional application No. 60/167,389, filed on Nov. 24, 1999.

(51) Int. Cl. C12Q 1/68; G01N 33/566; C07K 14/00

(52) U.S. Cl. 435/6; 436/501; 530/395

(57) ABSTRACT

The present invention is directed to nucleic acid molecules and polypeptides encoding a dsRNA receptor (dsRNA-R). The dsRNA-R contains a THD, interacts with the MyD88 adapter protein, and may bind to dsRNA. The present invention is also directed to antibodies against dsRNA-R and to methods of modulating an immune response and the methods of identifying compounds which bind to and/or modulate dsRNA-R.

### CROSS-REFERENCE TO RELATED APPLICATION

This application is a continuation application of U.S. application Ser. No. 11/350,151, filed Feb. 8, 2006, which is a continuation application of U.S. application Ser. No. 10/129,158, filed Jan. 29, 2003, now abandoned, which is the National Stage of International Application No. PCT/US00/41726, filed Nov. 1, 2000, which claims benefit under 35 U.S.C. §119(e) of provisional application 60/163,157, filed Nov. 2, 1999 and provisional application 60/167,389, filed Nov. 24, 1999, which applications are hereby incorporated by reference in their entireties.

### FIELD OF THE INVENTION

The present invention is related generally to immune responses and to the identification of a protein and pathway for signaling an immune response, and specifically to identification of a double-stranded RNA receptor (dsRNA-R).

### BACKGROUND

The innate immune system of mammals recognizes and responds to molecular features characteristic of pathogenic organisms. Various portions of the pathogen, such as surface proteins, particular cell wall components and certain nucleotide sequences, may be recognized and trigger a variety of immune responses. It has long been known that cells carry a variety of receptors and membrane bound proteins that recognize these foreign elements and trigger the cascade known as the immune response. Two broad classifications or types of responses are well known: humoral, or antibody-mediated immunity; and cell-mediated immunity. Certain pathogens or conditions may be effectively controlled by primarily an antibody-mediated reaction, while other conditions or pathogens require a vigorous cellular response to mediate a host defense.

Adjuvants are compounds which are capable of potentiating the innate immune response. Adjuvants can potentiate both humoral and cellular immunity. For some conditions or diseases such as, for example, those caused by the human immunodeficiency virus or hepatitis C virus, it is particularly desirable to increase the innate cell-mediated immune response by the administration of an adjuvant.

It is an aim of the invention to characterize and identify a dsRNA-R, to differentiate it from other proteins or receptors that may lead to similar immune responses, to develop adjuvants that comprise dsRNA-R, and develop useful methods that will allow the design of novel dsRNA-R ligands that can retain the immunostimulatory properties of dsRNA while having more desirable pharmacological properties.

### SUMMARY OF THE INVENTION

The present invention is directed to, in part, isolated nucleic acid molecules encoding dsRNA-R, or a fragment thereof, a nucleotide sequence complementary to at least a portion of the nucleotide sequence encoding dsRNA-R and a nucleotide sequence homologous to the nucleotide sequence encoding dsRNA-R, or a fragment thereof.

The present invention is also directed to recombinant expression vectors comprising any of the nucleic acid molecules described above and to host cells transformed therewith.

The present invention is also directed to a polypeptide, or polypeptides, or a complex of polypeptides encoding dsRNA-R, or a homolog or fragment thereof. Such a polypeptide can be prepared by introducing a recombinant expression vector comprising any of the nucleic acid molecules described above into a compatible host cell, growing the host cell under conditions which allow expression of the polypeptide, and isolating the polypeptide from the host cell.

The present invention is also directed to compositions comprising any of the nucleic acid molecules or polypeptides described above and an acceptable carrier or diluent.

The present invention is also directed to isolated antibodies which bind to an epitope on a polypeptide encoded by any of the nucleic acid molecules described above.

The present invention is also directed to kits comprising antibodies which bind to a polypeptide encoded by any of the nucleic acid molecules described above and a control antibody.

The present invention is also directed to methods of modulating an immune response in a mammal by administering to the mammal an amount of a compound which binds to the dsRNA-R and either activates or inhibits dsRNA activation of the dsRNA-R The present invention is also directed to methods of identifying a compound which binds to or modulates the activity of dsRNA-R by contacting dsRNA-R, or cells expressing dsRNA-R, with a compound, and determining whether the compound binds to or modulates the activity of dsRNA-R.

### DETAILED DESCRIPTION

The present invention is based upon the surprising discovery of the identification of a specific dsRNA-receptor (dsRNA-R). It is further based on the surprising discovery that the dsRNA-R, or complex containing the same, contains a Toll homology domain

(THD). Discovery of the presence of a THD within the receptor has permitted testing for the necessity of known Toll receptors for the dsRNA responses and the further characterization of the dsRNA-R. The present invention is directed to, inter alia, a dsRNA-R that can be a single polypeptide, or a complex of a plurality of polypeptides, and can optionally contain additional components such as, for example, polysaccharides, lipids, and the like. The dsRNA-R, or complex containing the same, preferably comprises a THD and interacts with the MyD88 adapter protein. Further, the dsRNA-R may bind to dsRNA.

The practice of the present invention will employ, unless otherwise indicated, conventional methods of virology, immunology, microbiology, molecular biology and recombinant DNA techniques within the skill of the art. Such techniques are explained fully in the literature. See, e.g., Sambrook, et al., Molecular Cloning: A Laboratory Manual (2nd Edition, 1989); DNA Cloning: A Practical Approach, Vols. I & II (D. Glover, ed.); Methods in Enzymology (S. Colowick and N. Kaplan eds., Academic Press, Inc.); Fundamental Virology, 2nd Edition, Vols. I & II (B. N. Fields and D. M. Knipe, eds.), Remington's Pharmaceutical Sciences, 18th Edition (Easton, Pa.: Mack Publishing Company, 1990); Handbook of Experimental Immunology, Vols. I-IV (D. M. Weir and C. C. Blackwell, eds., 1986, Blackwell Scientific Publications); Handbook of Surface and Colloidal Chemistry (Birdi, K. S., ed, CRC Press, 1997) and Seymour/Carrahers Polymer Chemistry (4th edition, Marcel Dekker Inc., 1996).

I. Definitions

As used herein, the term “dsRNA-R” refers to a double-stranded RNA-receptor, which can be a single polypeptide, or a complex of a plurality of polypeptides, and can optionally contain additional components such as, for example, polysaccharides, lipids, and the like. The dsRNA-R can be a protein involved in the dsRNA signaling cascade or can be a receptor for binding dsRNA.

As used herein, the term “activity” refers to any activity, or cascade of activities, that is associated with dsRNA binding or signaling.

As used herein, the term “about” means ±10% of the value it modifies. As used herein, the term “antibody” is meant to, without limitation, refer to complete, intact antibodies, Fab fragments and F(ab)₂ fragments thereof, and chimeric antibodies.

As used herein, the term “homologous” refers to nucleotide or amino acid sequences characterised by a sequence identity of at least about 70%, more preferably at least about 80%, more preferably at least about 90%, and most preferably at least about 95% to the entire nucleotide or amino acid sequence encoding dsRNA-R, or to at least a portion of dsRNA-R. Homologous amino acid sequences include those amino acid sequences encoding conservative amino acid substitutions. Sequence identity can be determined by, for example, the Gap program (Wisconsin Sequence Analysis Package, Version 8 for Unix, Genetics Computer Group, University Research Park, Madison Wis.), using the default settings, which uses the algorithm of Smith and Waterman (Adv. Appl. Math., 1981, 2, 482-489, which is incorporated herein by reference in its entirety).

As used herein, the term “modulate” means an increase or decrease in the amount, or effect of a particular activity or protein.

### II. Modes of Carrying Out the Invention

One aspect of the present invention is directed to nucleic acid molecules comprising novel nucleotide sequences encoding dsRNA-R. The nucleic acid molecules are preferably either RNA or DNA, but may contain both RNA and DNA monomers or peptide nucleic acid monomers. The nucleic acid molecule may be single stranded or double stranded. The monomers of the nucleic acid molecules can be linked via conventional phosphodiester bonds or modified bonds, such as, for example, phosphorothioate bonds, and the like. In addition, the sugar moieties of the monomers may be modified by, for example, addition of 2′ substitutions which help confer nuclease resistance and/or cellular uptake. The nucleic acid molecule can also comprise a nucleotide sequence complementary to at least a portion of the nucleotide sequence that encodes dsRNA-R. Preferably, the nucleic acid molecule comprises a nucleotide sequence complementary to the entire sequence, but can comprise a nucleotide sequence complementary to a portion of the entire sequence. The nucleic acid molecule can also comprise a nucleotide sequence homologous to the nucleotide sequence that encodes dsRNA-R and can be at least about 70% homologous, determined as above-mentioned, more preferably at least about 80% homologous, more preferably at least about 90% homologous, and most preferably at least about 95% homologous to the entire sequence encoding dsRNA-R or to any portion thereof.

A wide variety of alternative cloning and in vitro amplification methodologies are well known to those skilled in the art. Examples of these techniques are found in, for example, Berger et al., Guide to Molecular Cloning Techniques, Methods in Enzymology 152 Academic Press, Inc., San Diego, Calif. (Berger), which is incorporated herein by reference in its entirety.

Another aspect of the present invention is directed to vectors, or recombinant expression vectors, comprising any of the nucleic acid molecules described above. Vectors are used herein either to amplify DNA or RNA encoding dsRNA-R in order to express DNA which encodes dsRNA-R. Preferred vectors include, but are not limited to, plasmids, phages, cosmids, episomes, viral particles or viruses, and integratable DNA fragments. Preferred viral particles include, but are not limited to, adenoviruses, parvoviruses, herpesviruses, poxviruses, adeno-associated viruses, Semliki Forest viruses, vaccinia viruses, and retroviruses. Preferred expression vectors include, but are not limited to, pcDNA3 (Invitrogen) and pSVL (Pharmacia Biotech), pGEM vectors (Promega), pPROEXvectors (LTI, Bethesda, Md.), Bluescript vectors (Stratagene), pQE vectors (Qiagen), pSE420 (Invitrogen), and pYES2 (Invitrogen).

Preferred expression vectors are replicable DNA constructs in which a DNA sequence encoding dsRNA-R is operably connected to appropriate control sequences capable of effecting the expression of dsRNA-R in an appropriate host cell or organism. DNA regions are operably connected when they are functionally positioned with respect to each other. Control sequences include, but are not limited to, a promoter, an operator, a ribosomal binding sequence, and transcription/translation termination sequences.

Preferred vectors preferably contain a promoter which is recognised by the host organism. The promoter sequences of the present invention may be either prokaryotic, eukaryotic or viral. Examples of suitable prokaryotic sequences include the P_{R} and P_{L} promoters of bacteriophage lambda (The bacteriophage Lambda, Hershey, A.D., Ed., Cold Spring Harbor Press, Cold Spring Harbor, N.Y. (1973), and Lambda II, Hendrix, R. W., Ed., Cold Spring Harbor Press, Cold Spring Harbor, N.Y. (1980), each of which is incorporated herein by reference in its entirety); the trp, recA, heat shock, and lacZ promoters of E. coli and the SV40 early promoter (Benoist, et al. Nature, 1981, 290, 304-310, which is incorporated herein by reference in its entirety). Additional promoters include, but are not limited to, mouse mammary tumor virus, long terminal repeat of human immunodeficiency virus, maloney virus, cytomegalovirus immediate early promoter, Epstein Barr virus, rous sarcoma virus, human actin, human myosin, human hemoglobin, human muscle creatine, and human metalothionein. Moreover, appropriate expression vectors can include a marker which allows the screening of the transformed host cells. Expression vectors can be prepared by standard methodology.

Another aspect of the present invention is directed to transformed host cells having an expression vector comprising any of the nucleic acid molecules described above. Suitable host cells for expression of the polypeptides of the invention include, but are not limited to, prokaryotes, yeast, and eukaryotes. Suitable prokaryotic cells include, but are not limited to, bacteria of the genera Escherichia, Bacillus, Salmonella, Pseudomonas, Streptomyces, and Staphylococcus. Suitable eukaryotic cells include, but are not limited to, insect cells, HeLa cells, Chinese hamster ovary cells (CHO cells), African green monkey kidney cells (COS cells), and murine 3T3 fibroblasts.vvSuitable yeast cells include, but are not limited to, the genera Saccharomyces, Pichia, and Kluveromyces. The polypeptides of the invention can also be expressed using a baculovirus expression system (Luckow et al., Bio/Technology, 1988, 6, 47, Baculovirus

Expression Vectors: A Laboratory Manual, O'Rielly et al. (Eds.), W. H. Freeman and Company, New York, 1992, and U.S. Pat. No. 4,879,236,each of which is incorporated herein by reference in its entirety). In addition, the MAXBACJ complete baculovirus expression system (Invitrogen) can, for example, be used for production in insect cells. Propagation of such cells in cell culture is a routine procedure as described in, for example, Tissue Culture, Academic Press, Kruse and Patterson, eds. (1973), which is incorporated herein by reference in its entirety.

Another aspect of the present invention is directed to an isolated polypeptide encoded by a nucleic acid molecule described above. In preferred embodiments of the invention, the isolated polypeptide comprises an amino acid sequence encoding dsRNA-R. Alternatively, the polypeptide is a fragment of the polypeptide encoding dsRNA-RP. Alternatively, the polypeptide comprises an amino acid sequence homologous to dsRNA-R or a fragment thereof. A polypeptide having an amino acid sequence which has at least about 70% sequence identity or homology, determined as above-mentioned, at least about 80% sequence identity or homology, preferably about 90% sequence identity or homology, more preferably about 95% sequence identity or homology and most preferably about 98% sequence identity or homology to dsRNA-R is contemplated as being included in the present invention. A preferred homologous polypeptide comprises at least one conservative amino acid substitution compared to native dsRNA-R. Other preferred homologous polypeptides comprises two, three, four, five, six, seven, eight, nine, or up to ten conservative amino acid substitutions compared to native dsRNA-R The polypeptides can be expressed in host cells as fusion proteins which may include regions from heterologous proteins. The polypeptides of the invention also may include regions from the same protein but which differ from the naturally-occurring polypeptide in sequence. In addition, homologous dsRNA-R polypeptide comprises those polypeptides having at least about 70% functional homology, at least about 80% functional homology, preferably about 90% functional homology, more preferably about 95% functional homology and most preferably about 98% functional homology compared to dsRNA-R, respectively. Thus, it is to be understood that the present invention includes proteins homologous to, and having essentially at least one biological property (functional homology) that is substantially similar to a biological property of dsRNA-R.

The polypeptides of the invention encoding dsRNA-R can be isolated, for example, by screening recombinant expression libraries or sequence databases, or the like, for the ability to bind dsRNA, for comprising a THD domain, and for the ability to interact with MyD88 adapter protein. The MyD88 adapter protein is required for signaling from receptors for products, such as lipopolysaccharides and lipoproteins, as well as for interleukin-1. The polypeptides of the present invention are preferably provided in an isolated form, are preferably substantially purified, and most preferably are purified to homogeneity. Host cells are preferably lysed and the polypeptide is recovered from the lysate of the host cells. Alternatively, the polypeptide is recovered by purifying the cell culture medium from the host cells, preferably without lysing the host cell. The polypeptides can be recovered and purified from recombinant cell cultures by well-known methods, including ammonium sulfate or ethanol precipitation, anion or cation exchange chromatography, hydroxylapatite chromatography and lectin chromatography.

Another aspect of the present invention is directed to compositions, including pharmaceutical compositions, comprising any of the nucleic acid molecules or polypeptides described above and an acceptable carrier or diluent. Preferably, the carrier or diluent is pharmaceutically acceptable. The carrier or diluent can include any. sustained release material known in the art, such as glyceryl monostearate or glyceryl distearate, alone or mixed with a wax. The formulations can also include wetting agents, emulsifying and suspending agents, preserving agents, sweetening agents, thickeners or flavoring agents. The formulations of the invention can be formulated so as to provide quick, sustained, or delayed release of the active ingredient after administration to the patient by employing procedures well known in the art. The pharmaceutical compositions can be sterilized and mixed, if desired, with auxiliary agents, emulsifiers, salt for influencing osmotic pressure, buffers and/or coloring substances, and the like, that do not deleteriously react with the active compounds. Thickeners, flavorings, diluents, emulsifiers, dispersing aids or binders can also be added.

The polypeptides of the invention can be used to generate antibodies against the same and used to screen for compounds that modulate the activity of dsRNA-R or dsRNA. Preferably, the antibody binds to an epitope within dsRNA-R. The antibodies can be monoclonal or polyclonal. Hybridomas which produce antibodies that bind to the polypeptides of the invention, and the antibodies themselves, are useful in the isolation and purification of the polypeptides. In addition, antibodies may be specific inhibitors of dsRNA-R activity. Antibodies that specifically bind to the polypeptides of the invention can be used to purify the protein from natural sources or through recombinant technology using well known techniques and readily available starting materials. Methods of making antibodies are known to persons skilled in the art. For techniques for preparing monoclonal antibodies, see e.g. Stiites et al (eds.), Basic and Clinical Immunology (4th ed), Lange Medical Publications, Los Altos, Calif., which is incorporated herein by reference in its entirety. Production of antibodies, Fab fragments and F(ab)₂ fragments are described in, for example, Harlow, E. and D. Lane (1988) Antibodies: A Laboratory Manual, Cold Spring Harbor Laboratory, Cold Spring Harbor, N.Y. which is incorporated herein by reference.

The present invention is also directed to kits, including pharmaceutical kits. The kits can comprise any of the nucleic acid molecules, polypeptides, or antibodies described above, as well as appropriate controls. The kit preferably comprises additional components, such as, for example, instructions, solid support, reagents helpful for quantification, and the like.

Another aspect of the present invention is directed to methods of modulating an immune response in a mammal by administering to the mammal an amount of dsRNA-R, antibody to dsRNA-R, or a compound that binds to dsRNA-R. The amount will be dependent on the animal species, size of the animal, and the like but can be determined by those skilled in the art. The route of administration can be any route that effectively transports the active compound to the appropriate or desired site of action, such as oral, nasal, rectal, pulmonary, transdermal or parenteral, subcutaneous, intravenous, intraurethral, intramuscular, intranasal, ophthalmic solution, or an ointment, the parenteral or the oral route being preferred.

Another aspect of the present invention is directed to methods of identifying compounds which bind to either nucleic acid molecules or polypeptides encoding dsRNA-R comprising contacting dsRNA-R, or a nucleic acid molecule encoding the same, with a compound, and determining whether the compound binds dsRNA-R, or a nucleic acid molecule encoding the same. Binding can be determined by binding assays which are well known to those skilled in the art, including, but not limited to, gel-shift assays, Western blots, radiolabeled competition assays, co-fractionation by chromatography, co-precipitation, ELISA, and the like, which are described in, for example, Current Protocols in Molecular Biology, 1999, John Wiley & Sons, NY, which is incorporated herein by reference in its entirety. The dsRNA-R polypeptide or nucleic acid molecule used in such a test may either be free in solution, attached to a solid support, attached to a cell surface or located within the cell.

Another aspect of the present invention is directed to methods of identifying compounds which modulate signaling activity of dsRNA-R comprising contacting dsRNA-R with a compound, and determining whether the compound modifies activity of dsRNA-R. The activity in the presence of the test compound is measured and compared to the activity in the absence of the test compound. Where the activity of the sample containing the test compound is higher than the activity in the sample lacking the test compound, the compound will have increased activity. Where the activity of the sample containing the test compound is lower than the activity in the sample lacking the test compound, the compound will have inhibited activity. The dsRNA-R used in such a test can either be free in solution in the presence of suitable substrates, attached to a cell surface, or located within a cell.

Compounds which bind to and/or modulate dsRNA-R have utility in, for example, vaccine adjuvants promoting cell-mediated immune responses, antibacterials (e.g. protection from Listeria infection), tumor immunotherapy, allergy treatment (e.g. suppressing IgE in human PBMC, shifting from Th2 to Th1), and as anti-inflammatory agents (e.g. for use in cystic fibrosis, sepsis, heart disease, chlamydia, inflammatory bowel disease, arthritis, and multiple sclerosis).

The invention is further illustrated by way of the following examples which are intended to elucidate the invention. The foregoing examples are meant to illustrate the invention and are not to be construed to limit the invention in any way. Those skilled in the art will recognize modifications that are within the spirit and scope of the invention. It is intended that all references, including each of the patents, applications, and printed publications, mentioned herein be hereby incorporated by reference in their entirety.

### III. EXAMPLES

### Example 1

### General Methodology

Animals and Cell Lines

Bone marrow was harvested from female mice 6-12 weeks old of various genotypes (C57B1/6, Balb/c; Charles River; or C3H/HeJ, Jackson Labs). Bone marrow cells were used fresh or were frozen in fetal calf serum (FCS; Summit) containing 10% dimethylsulfoxide and stored at −80° C. Bone-marrow macrophages (BMMO) were prepared as described in Current Protocols in Immunology, supra. Briefly, fresh or frozen bone marrow cells were cultured in RPMI, 10% heat inactivated (30 minutes, 56° C.) fetal calf serum (FCS), 2 mM L-glutamine, 100 μg/ml streptomycin, 100 units/ml penicillin, 50 μM β-mercaptoethanol and 100 U/ml recombinant macrophage colony stimulating factor (M-CSF; R&D Systems). After 24 hours, nonadherent cells were removed to a new dish, and culture continued for 7 days to produce a macrophage monolayer. Nonadherent BMDDCs were produced by culture in the same medium supplemented with GM-CSF (200 U/ml; Preprotech) rather than M-CSF. The RAW 264.7 mouse macrophage cell line (originally derived from a Balb/c mouse) was obtained from the American Type Culture Collection (ATCC). RAW 264.7 cells are cultured in DMEM with 10% heat inactivated FCS, L-glutamine, streptomycin, penicillin, and 1 μM sodium pyruvate.

Reagents

Cell cultures were treated with the following reagents: K235 E. coli LPS, gel filtration purified (Sigma), monophosphoryl lipid A from S. minnesota R595 (MPL; RIBI Immunochem Research, Inc.), sonicated 10 minutes prior to addition. Oligonucleotides with phosphorothioate backbones were synthesized by Oligos Etc (Wilsonville, Oreg.). dsRNA can be synthesized according to current technology known by those skilled in the art. Recombinant mouse interleukin-1β (IL-1) (Endogen) and interleukin-18 (IL-18) (Biosource) were also used to stimulate RAW264.7 cells in some experiments.

Immunoblotting

Following stimulation, BMMO or RAW 264.7 cells were lysed in 1% Triton X-100, 50 mM Tris, 62.5 mM EDTA (pH 8.0) with CompleteJ protease inhibitor cocktail (Boehringer Mannheim) (Triton lysis buffer). Lysates were boiled in reducing sample buffer, separated on 10% polyacrylamide gels using the NUPAGEJ Bis-Tris electrophoresis system (Novex). Nitrocellulose membranes were probed with antibodies against IκB-α, phosphorylated IκB-α, (New England BioLabs), or anti-IL-18 (Santa Cruz Biotechnology) according to manufacturer's instructions, and visualized with enhanced chemiluminescence (Amersham).

κB-Luc Assay

RAW 264.7 cells were seeded into 6-well plates (Corning) at a density of 3×10⁵ cells per well 24 hours prior to transfection. Plasmids used in transfection were pNF-κB-Luc (Clontech) and pCR3.V64-Met-Flag-MyD88lpr (kindly provided by Jurgen Tschopp; described in (Burns et al., J. Biol. Chem., 1998, 273, 12203-9). Total plasmid DNA concentration was normalized across all wells by addition of empty vector (pCMVKm2, Chiron). Cells were transfected with stated concentrations of DNA in Opti-MEM I (Gibco BRL) with 10 ml of LIPOFECTAMINE (Gibco BRL) per well according to manufacturer's instructions. Cells were incubated with the transfection mixture 3 hours at 37° C., then culture media was replaced and cells were allowed to recover overnight. The following day transfected cells were treated in culture media at 37° C., 5% CO₂ with phosphorothioate oligonucleotides, MPL, LPS, or cytokines at indicated concentrations and times. Cells were washed once with cold phosphate buffered saline (PBS), and lysed with Reporter Lysis Buffed (Promega). Luciferase activity in lysate supernatants was determined using Microlite 2 plates (Dynex) and a ML 3000 Luminometer (Dynatech) all according to manufacturer's instructions. For detection of FLAG-MyD88lpr expression, the insoluble pellet from transfected RAW 264.7 cells was resuspended in Triton lysis buffer and sonicated 10 minutes prior to addition of sample buffer and boiling. After separation and transfer to nitrocellulose, membranes were immunoblotted using FLAG M2, (Sigma) antibody.

Flow Cytometry BMDDC were treated overnight with adjuvants as indicated, washed, resuspended in cold PBS/2% FBS containing FcBlock (0.25 μg/10⁶ cells; Pharmingen), and FITC-and PE-conjugated antibodies (1 μg/10⁶; cells) were added 5 minutes later. Cells were incubated with antibodies on ice 30 minutes, washed, and analyzed by flow cytometry on a FACScan (Becton-Dickenson). Only cells from BMDDC cultures staining positive for CD11c and having forward-and side-scatter properties of live cells were included in the analysis. Changes in CD86 expression are assessed based on geometric mean fluorescence of CD86-FITC staining of the live CD11c cells treated with adjuvants normalized to the geometric mean CD86-FITC fluorescence of untreated BMDDC cultures.

### Example 2

### Expression of A Dominant-Negative MyD88 Blocks dsRNA Induction of κB-Dependent Reporter Gene

RAW 264.7 cells were cotransfected with the κB-luciferase plasmid and an expression vector encoding a dominant-negative form of MyD88, an adapter protein required for signaling by members of the Toll/IL-1 receptor family. The dominant-negative MyD88 used in these experiments, MyD88lpr, has an intact THD, but contains a point mutation in the death domain (DD). A similar mutation in the death domain of Fas in the lpr mouse abrogates Fas signaling, presumably by altering conformation of the death domain, thus blocking association with downstream signaling molecules. Over-expression of MyD88lpr in RAW 264.7 cells is expected to interrupt MyD88-dependent signaling by competing with endogenous MyD88 for association with proteins containing Toll homology domains. This effect of MyD88lpr is specific to Toll-related proteins, as the mutation in the death domain should prevent association with downstream signaling components that might be shared with other activators of NF-κB.

Transfection of RAW 264.7 cells with the plasmid encoding FLAG-tagged MyD88lpr resulted in expression of a protein of the expected size, as determined by immunoblotting with an Anti-FLAG antibody (data not shown). RAW 264.7 cells were then transfected with κB-Luc (1 μg) alone or with a plasmid encoding MyD88lpr at two different ratios, 10:1 or 1:1. Total plasmid DNA concentration was normalized across all wells by addition of empty vector. Twenty-four hours post-transfection, cells were treated with dsRNA (25 μg/ml; poly (dI:dC)), monophosphoryl lipid A (MPL) (1 μg/ml) or LPS (1 μg/ml). In RAW 264.7 cells expressing the dominant-negative MyD88lpr inhibited κB-dependent luciferase activity induced by MPL or LPS. In a similar manner, cells which were cotransfected with the κB-luciferase plasmid and an expression vector encoding a MyD88lpr inhibited κB-dependent luciferase activity induced by dsRNA treatment. These results demonstrate MyD88 function is required for full activation of NF-κB by dsRNA. Since all known receptors that require MyD88 share a common structural feature, THD, this finding also implied that a component of the putative dsRNA receptor-has a THD. The presence of a receptor with a THD and involvement of Toll pathway components in signaling induced by dsRNA is novel.

### Example 3

### Requirement for TLR4 in dsRNA Signal Transduction

A preferred method of determining the polypeptide sequence of dsRNA-R is to test whether known TLRs might be likely candidates. A requirement for TLR4 for responses to LPS in mouse has been demonstrated using the endotoxin unresponsive strains C3H/HeJ which has a point mutation in TLR4, and C57B1/1OScCr and C57B1/1OScNCr mice, which do not express TLR4 (Vogel et al., J. Immunol., 1999, 162, 5666-70, Qureshi et al., J. Exp. Med., 1999,189,615-25, Chow et al., J Biol. Chem., 1999, 274,10689-92, Hoshino et al., J. Immunol., 1999, 162, 3749-52, and Poltorak et al., Science, 1998, 282, 2085-8).

To assess the role of TLR4 in responses of APCs to dsRNA and MPL in vitro, BMDDC from LPS-responsive mice (Balb/c) and C3H/HeJ mice are cultured overnight with these adjuvants and upregulation of markers of DC activation/maturation is assayed. Cell-surface CD86 expression is quantified by flow cytometry. CD86 is a NF-κB target gene upregulated in activated dendritic cells and macrophages, which enhances their capacity to activate antigen-specific T cells. BMDDC from wild-type (Balb/c) and TLR4 mutant mice (C3H/HeJ) are grown in GM-CSF for 6 days and then treated overnight with dsRNA (5 μM), a negative control oligonucleotide (5, μM), or LPS (1 μg/ml), or left unstimulated. Cell surface expression of CD86 on live CD11c positive cells can be assayed by flow cytometry. Results can be shown as the geometric mean fluorescence (MF) of adjuvant-treated BMDDC normalized to MF of untreated BMDDC from the same culture. If wild-type BMDDC and TLR4 mutant BMDDC treated with dsRNA show increased cell-surface CD86 expression, TLR4 may not be required for APC activation in vitro by dsRNA.

Further experiments similar to the one above can be carried out to test other known Toll-like receptors to better characterize the precise nature of the dsRNA-R. This can be carried out in any comparable cell lines having known intact TLRs and mutations that render those receptors non-functional. Identification of the cells with a receptor that is activated by dsRNA when intact, but not activated when the receptor is defective, will pinpoint precisely which TLR and which particular THD is present.

The results disclosed herein also will be recognized by those skilled in the art to provide useful approaches for developing compounds that may serve as agonists or antagonists of cellular signaling that is mediated by receptors which require the adapter protein MyD88 for their signaling pathways. Compounds can be incubated with cells and assayed to see if the cascade of events known to follow from dsRNA activation has been effected. The cells can then be transfected with the MyD88lpr gene. Compounds that produce the effect in controls without the MyD88lpr gene, but fail to do so when the MYD88lpr gene is expressed will have been shown to activate intracellular signaling via pathways which require the MyD88 adapter protein.

### Example 4

### Isolation of dsRNA-R

A number of different procedures can be utilized to identify the Toll-related, dsRNA-R component. First, one skilled in the art can examine the ability of known and novel Toll-like receptors to confer responsiveness to dsRNA on unresponsive cells. Second, one skilled in the art can use MyD88 to purify interacting proteins specifically activated by dsRNA and not other Toll receptor ligands, (e.g. LPS), or to isolate the corresponding CDNA using the yeast two-hybrid system.

Mouse Toll-like receptors 1-6 (TLR1-6) have closely related human homologs. Complete or partial sequences are available in public databases for the mouse genes. Expression vectors encoding the TLRs can be transfected into a dsRNA unresponsive cell line (e.g. NIH3T3) along with a reporter gene activated by dsRNA (e.g. κB-luciferase). If any of the cotransfected TLRs confer responsiveness to dsRNA, that will be reflected by expression of the reporter gene upon treatment with dsRNA. A similar approach can be used to test the involvement of any candidate dsRNA-R component in dsRNA signaling, including novel TLRs. Approaches to identifying and cloning novel Toll-related receptors include genomics, screening sequence databases, degenerate PCR, the yeast two-hybrid system, and library screening by hybridization.

MyD88lpr, which contains an intact Toll homology domain and a single inactivating point mutation in the death domain can be expressed as a recombinant protein, either in bacteria or in eukaryotic cells. The recombinant protein can be used as an affinity reagent to isolate and purify interacting proteins from dsRNA-treated cells (e.g. RAW264.7). Binding of downstream effector molecules in the TLR signaling pathway (e.g. IRAK, TRAF6) should be minimized or eliminated by the “lpr” mutation in the death domain. Thus, the protein(s) binding specifically to MyD88lpr will be those that interact with the THD and/or the region linking the THD to the death domain. As Toll homology domains associate via homotypic interaction, the dsRNA-R component containing a THD is expected to bind the MyD88 THD. Once the putative dsRNA-R has been affinity purified on MyD88, the corresponding cDNA can be isolated by standard methods such as, for example, peptide analysis and PCR. Similarly, MyD88lpr can be used as bait in the yeast two-hybrid system to isolate cDNAs encoding interacting proteins. The relevance of these cDNAs to dsRNA oligonucleotide-induced signaling can then be tested as described above.

What is claimed is:

1. A method of identifying a compound which binds to a Toll-like receptor that binds dsRNA or modulates the ability of said receptor to bind dsRNA, wherein said receptor comprises a Toll Homology Domain that binds to MyD88, the method, comprising:
contacting said receptor, or cells expressing said receptor, with a compound; and
determining whether said compound binds to or modulates the ability of said receptor to bind dsRNA.

2. A method of forming a receptor/ligand complex, the method comprising contacting a Toll-like receptor that binds dsRNA and that comprises a Toll Homology Domain that binds to MyD88, with a dsRNA under receptor/ligand binding conditions.
